(19) United States
(12) Patent Application Publication
(10) Pub. No.: US 2004/0167529 A1
(43) Pub. Date: Aug. 26, 2004

(54) Device and method for collecting surgical material

(75) Inventors: Lew Papendick, Rapid City, SD (US); Thomas Blue, Rapid City, SD (US)

Correspondence Address: WESTMAN CHAMPLIN & KELLY, P.A., SUITE 1600 - INTERNATIONAL CENTRE, 900 SECOND AVENUE SOUTH, MINNEAPOLIS, MN, 55402-3319, US

(73) Assignee: Six-O, Ltd., Rapid City, SD

(21) Appl. No.: 10/401,059

(22) Filed: Mar. 27, 2003

Related U.S. Application Data
(60) Provisional application No. 60/449,755, filed on Feb. 24, 2003.

(51) Int. Cl. A61F 5/00

(52) U.S. Cl. 606/86

(57) ABSTRACT

The present invention includes a collection device for collecting material during a surgical procedure. In one aspect, the collection device includes a collection chamber having an opening sufficient for permitting collection of the material during the surgical procedure and a scoop wall extending from the encompassing wall, the scoop wall having a distal end portion for positioning proximate a source of the material and for guiding the material to the collection chamber. In another aspect of the invention, the collection chamber has at least one aperture within the collection chamber of a size to permit flow of fluid from the chamber when the collected material is contained within the chamber. Fluid may also be poured out of the chamber without the collected material following the fluid thereby retaining the collected material within the chamber. In yet another aspect, the collection device is used for collecting bone chips that are produced during the surgical procedure and the device includes a wall extending from the collection portion of the invention that is sufficiently pliant to generally conform to the operative site from which the bone chips are being collected. Yet another aspect of the present invention includes a collection chamber that has a wall structure with a front and a back wall and an opening for collecting bone chips positioned between the front and the back wall with the front wall having a cutout positioned proximate the top portion of the front wall of a size sufficient for positioning a reamer therein such that bone chips produced by the reamer fall within the collection chamber. Another aspect of the present invention includes a smooth, concave inner surface within the chamber for facilitating the effective retrieval of bone chips.

### CROSS-REFERENCE TO RELATED APPLICATION(S)

None.

### BACKGROUND OF THE INVENTION

The present invention relates to the field of surgery, and particularly, to the field of knee surgery. More specifically, the present invention relates to a device for the harvesting of bone reamings produced during anterior cruciate ligament reconstruction surgery.

One of the more commonly performed procedures in knee surgery is reconstruction of the anterior cruciate ligament. These reconstructions are almost always done arthroscopically. One of the problems faced in anterior cruciate ligament knee surgery is that defects are created in the bone during surgery. One such defect is called the patellar defect.

For several reasons, bone reamings resulting from the surgery provide the most desirable material for treating a bone defect. The reamings provide greater surface area of bone, which promotes healing. The reamings are more pliable than other bone material and therefore, mold well to the shape of the patellar defect. Other types of grafting material such as bone core and curetting tibial bone have time, difficulty, and cost disadvantages. It takes more time for a surgeon to use a bone core because it is rigid and has to be sized, cut and formed to fit the defect. It is also more difficult for a surgeon to work with and is more costly because of the additional operating room and staff time needed. Due to a bone core's rigidity, even after taking the time to size, cut and form the bone, it does not fit the defect as well as reamings. In addition to the same time, difficulty, and cost disadvantages of using a bone core, curetting tibial bone from the tibial defect at the harvest site creates further morbidity to the tibia. Additionally, autogenous grafting material provides the most optimal healing and effective recovery for the patient.

It has been known to use a stiff metal medicine cup to attempt to collect bone chips from reamings for use as grafting material in anterior cruciate ligament reconstruction surgery.

### BRIEF SUMMARY OF THE INVENTION

The present invention includes a collection device for collecting material during a surgical procedure. In one aspect, the collection device includes a collection chamber having an opening sufficient for permitting collection of the material during the surgical procedure and a scoop wall extending from the encompassing wall. The scoop wall has a distal end portion for positioning proximate a source of the material and for guiding the material to the collection chamber.

In another aspect of the invention, the collection chamber has at least one aperture within the collection chamber of a size to permit flow of fluid from the chamber when the material being collected is contained within a fluid. The collected material is retained in the chamber.

In yet another aspect, the collection device is used for collecting bone chips that are produced during the surgical procedure and the collection chamber is defined by a wall having an outer surface of a configuration that generally conforms to the body proximate to the surgical site from which the bone chips are being collected and the scoop wall has a surface that is positionable proximate a source of the bone chips such that the bone chips are guided by the scoop wall to the chamber.

Yet another aspect of the present invention includes a collection chamber that has a wall structure with a front and a back wall and an opening for collecting bone chips positioned between the front and the back wall with the front wall having a cutout positioned proximate the top portion of the front wall of a size sufficient for positioning a reamer therein such that bone chips produced by the reamer fall within the collection chamber.

### BRIEF DESCRIPTION OF THE DRAWINGS

FIG. 1 is a perspective view showing the front and side of the present invention.

FIG. 2 is a sectional view taken along the line 2-2 in FIG. 4.

FIG. 3 is a perspective view illustrating the present invention in use.

FIG. 4 is a top view of the present invention.

FIG. 5 is a perspective view of the device on a surface in a resting position.

### DETAILED DESCRIPTION

The present invention allows for collection of surgical materials, such as bone reamings during anterior cruciate ligament (ACL) knee surgery. The collected reamings may later be used as autogenous graft material.

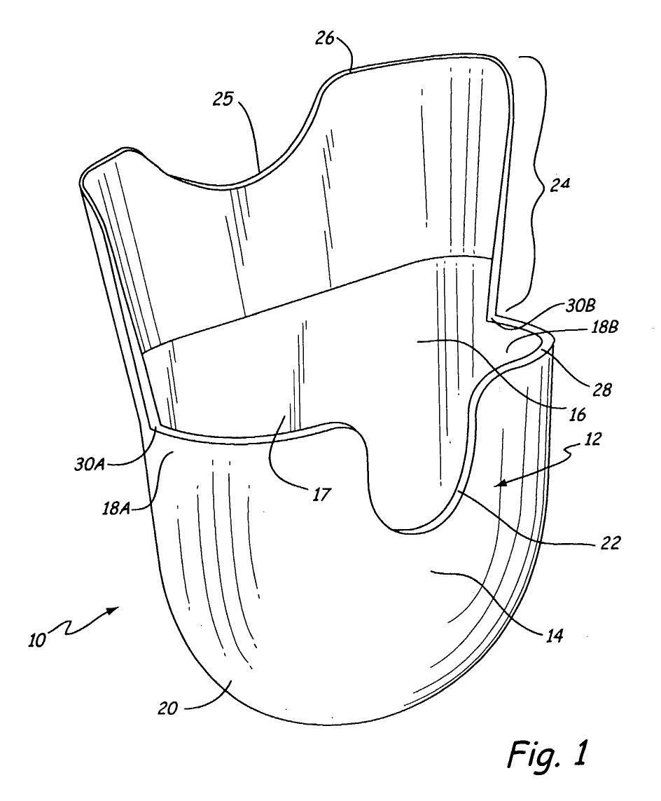

Bone reamings (chips) created during surgery are generally not utilized or are underutilized as autogenous bone graft material because there are no current generally accepted collection methods that allow for efficient and effective collection and separation of the reamings. A collection device of the present invention generally indicated at 10 in FIG. 1, allows a surgeon to readily gather, separate, and apply bone reamings as graft material during surgery.

The collection device 10 is comprised of a collection chamber 12 and a scoop wall 24. The collection chamber 12 is formed from a front face 14 and a back face 16 defining an opening 17. The front face 14 and the back face 16 are connected at sides 18A, 18B, and bottom 20. As illustrated in the exemplary embodiment, the connection between the front face 14 and the back face 16 is seamless and forms an encompassing wall that defines the collection chamber 12. The upper center of the front face 14 has a cutout 22 that facilitates the surgeon's use of a reamer. The cutout on the front of the device allows it to be positioned on the patient's leg below the site where the reamer enters the body to create the tibial tunnel while not interfering with the reamer positioning or the proper angle the surgeon needs for correctly creating the tibial bone tunnel. In the exemplary embodiment, the cutout 22 is approximately 0.830 inch wide and 0.788 inch in length, which is about twice the size of a typical reamer used in ACL reconstruction surgery. The cutout being larger than the diameter of the reamer permits angular positioning and repositioning if needed of the reamer in the cutout.

The back face 16 of the collection chamber 12 is extended and forms the scoop wall 24. The scoop wall 24 serves as a collection surface for the collection chamber 12, in which the scoop wall 24 functions as a slide, scoop or funnel. The dimensions of the scoop wall 24 can vary. Additionally, in the exemplary embodiment, the back face 16 is integral with the scoop wall 24 as one continuous piece. Variations of the extended rear surface of the collection chamber 12 can include the back face 16 and the scoop wall 24 as two separate pieces which are then connected or attached to form the extended back surface.

The scoop wall 24 also includes a depression 25 disposed centrally at a top edge 26 which allows for better positioning of the reamer and the scoop wall 24 against the leg. The scoop wall 24 includes a central portion and sidewall portions 30A and 30B. The sidewall portions 30A and 30B extend outwardly from the central portion and curve forwardly to form a channel to guide bone reamings to the collection chamber 12. In this exemplary embodiment, the scoop wall 24 is approximately 1.394 inches in length (when measured from the top edge 26 of the scoop wall 24 to the collection chamber top edge 28) not taking into account the depression which is approximately 1.0 cm and approximately 2.572 inches in width.

The scoop wall 24 is tapered such that a portion closer to the collection chamber 12 has the same thickness as the collection chamber 12, which is approximately 0.062 inch in the exemplary embodiment. The thickness of scoop wall 24 begins to taper off approximately one third of the way distal from the defined back face 16 of the collection chamber 12 to a final thickness of approximately 0.025 inch at a top edge 26. The tapered surface of the scoop wall 24 allows for the top portion of scoop wall 24 to be more flexible to conform to the patient's leg below the operative site.

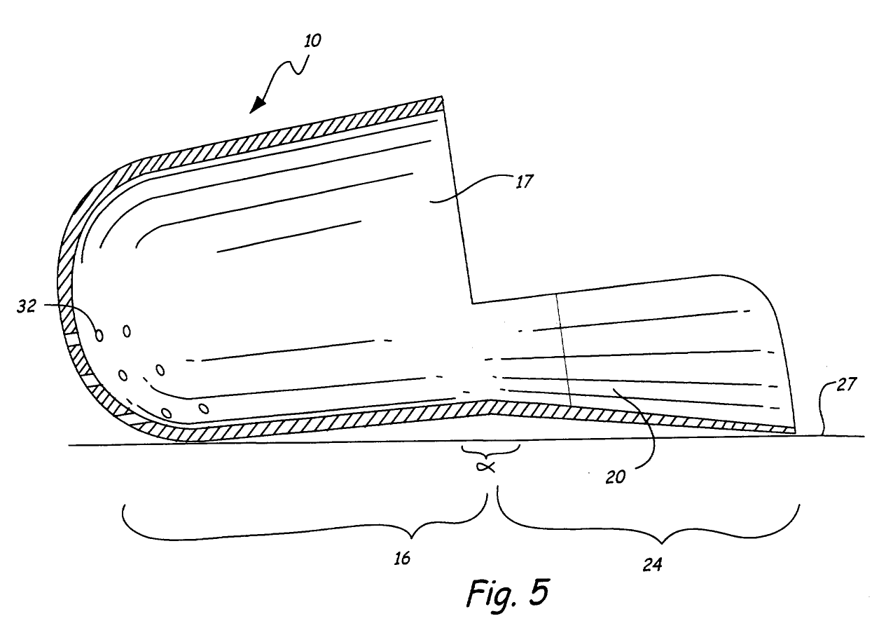

The scoop wall 24 forms an obtuse angle α with the back face 16 of the collection chamber 12 so that when the device 10 is placed on a surface 27 such as a table, the bone chips are retained within the chamber 12 as is seen in FIG. 5. When placed on a surface, the collection chamber 12 is tilted upwardly, to retain the bone chips.

On the lower portion of the back face 16 of the collection chamber 12 are openings 32 through which fluid drains from the collection chamber 12. In the exemplary embodiment, there are eighteen openings 32 that are approximately 0.12 inches each in diameter. The openings 32 are set apart approximately 0.25 inches horizontally and approximately 0.187 inches vertically. The openings 32 are positioned in three rows with five openings 32 in the row closest to the back face 16, six openings 32 in the middle row and seven openings 32 in the row closest to the front face 14. However, the openings 32 can be located anywhere within the collection chamber and in any formation so long as fluid continues to drain from the chamber when the device is held or is placed on a surface. When the chamber is filled with bone chips, the bone chips may stop fluid from flowing from the chamber through the openings 32. The device may then be pressed against or placed on a sponge to wick away further liquid from the chamber before the bone chips are used as explained subsequently below.

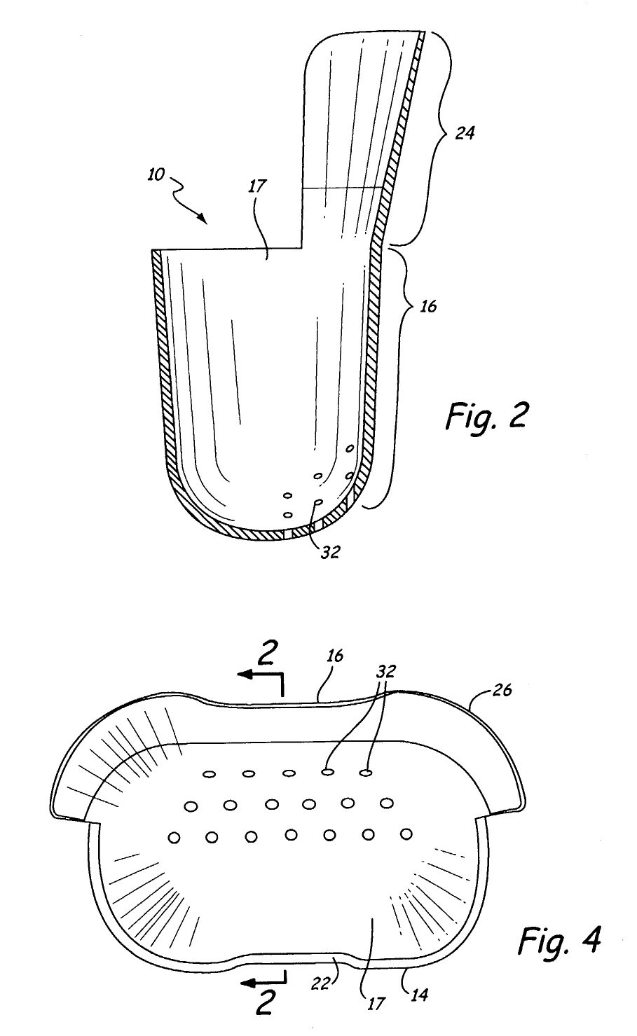

When used during surgical procedures, the collection device 10 is placed below a reamer 44 with the scoop wall 24 pressed against the leg just below the operative site. While the reamer 44 is being used to create a tunnel in the bone, the lower portion of the reamer 44 can be maneuvered because of the space created by the cutout 22 in the upper front face 14 of the collection chamber 12. Bone reaming expelled from the tunnel slide down the scoop wall 24 and fall into the collection chamber 12. The bone reamings settle to the bottom of the collection chamber. Some saline or other fluids are then slowly poured from the device over 18A or 18B and the remaining fluids are allowed to drain from the collection device 10 through openings 32 in collection chamber 12. As illustrated in FIGS. 2 and 4, the openings 32 are of a size to provide efficient draining of fluids while retaining the majority of the reamings. In addition, the collection device is of a sufficient width that when positioned below the operative site a major portion of the bone reamings that are generated are deposited within the collection device. Although a collection device having a substantially cylindrical configuration will collect bone reamings, a device having a substantially oval or rectangular configuration and whose backwall can be placed to conform to the surface next to the operative site and which will conform to a surface of the operative site collects a major portion of the bone reamings being generated.

To understand the context in which the present invention is used, the following is a description of the surgical procedure. The order of some of these “steps”, especially 5 and 6, can be changed and often are depending on when the bone-tendon-bone (B-T-B) graft is ready. To begin the procedure, a patient is placed in a supine position with a tourniquet on the operative proximal thigh. The thigh is placed in a leg holder with the foot of the bed down at 90 degrees, allowing the knee to bend 90 degrees. The leg is then prepped and draped in the routine sterile fashion. A superolateral portal is placed just proximal and lateral to the patella, and the inflow cannula is placed in the portal. Saline is pumped into the knee with the inflow pump. The standard anterolateral and anteromedial portals are established. Once the portals are established, the procedure can be divided into six basic steps: 1. Debride the torn ACL and perform an intercondylar notchplasty, 2. Harvest B-T-B Graft from patella/tibia, 3. Ream tibial tunnel and collect reamings, 4. Ream femoral tunnel, 5. Pass and secure B-T-B ACL graft (that surgeon harvested from the patella/patella tendon/tibia), and 6. Place reamings in patellar defect and close peritenon. The order of the steps are interchangeable.

First, torn fragments of the ACL are débrided with a standard arthroscopic shaver through the anteromedial portal. The notchplasty is then performed, taking bone from the lateral and superior edge of the intercondylar notch of the femur. An arthroscopic burr is used to abrade bone and soft tissue in the intercondylar femoral notch until the femoral attachment of the ACL is visualized. The burr is then used to make a small divot approximately 2-3 mm from the over-the-top position at either the 11 o'clock (right knee) or 1 o'clock (left knee) position in the intercondylar notch of the femur.

Second, a skin incision is made adjacent to the patellar tendon. It is carried through the subcutaneous tissue to the peritenon. Retraction of skin and subcutaneous tissue is accomplished to visualize the entire patellar tendon from the tibial tubercle to the patella. The peritenon is then split midline and stripped from the patella and patellar tendon to give full visualization of the patellar tendon in its medial to lateral width. The central one-third of the patellar tendon is then harvested.

Using a powered saw blade, the patella is cut at approximately a 60 degree angle both on the medial and lateral sides. A slightly angled cut is also made on the cephalad surface. An osteotome is then used to wedge the pie-shaped piece of bone out of the patella. On the distal end similar saw cuts are made in the tibia and the autogenous graft is then lifted. At this point, the surgeon has obtained a fragment of the patellar bone, a piece of patellar tendon and a fragment of the tibial tubercle. The wound is then thoroughly irrigated.

Third, with electrocautery, an incision is made on the tibia just proximal to the attachment of the pes anserine tendons, midway between the anterior and posterior edge on the medial side of the tibia. The area is stripped of its periosteum to give access for the tibial guide used to produce the tibial tunnel.

With the arthroscope in the anterolateral portal, the tibial guide is then placed in the anteromedial portal. A guidepin is drilled from the external surface of the tibia to the above-mentioned site intra-articularly. Generally, the tibial guide is placed so that the guidepin of the tibial guide will exit intra-articularly in the footprint of the ACL at approximately the junction of the attachment of the posterior edge of the anterior horn of the lateral meniscus.

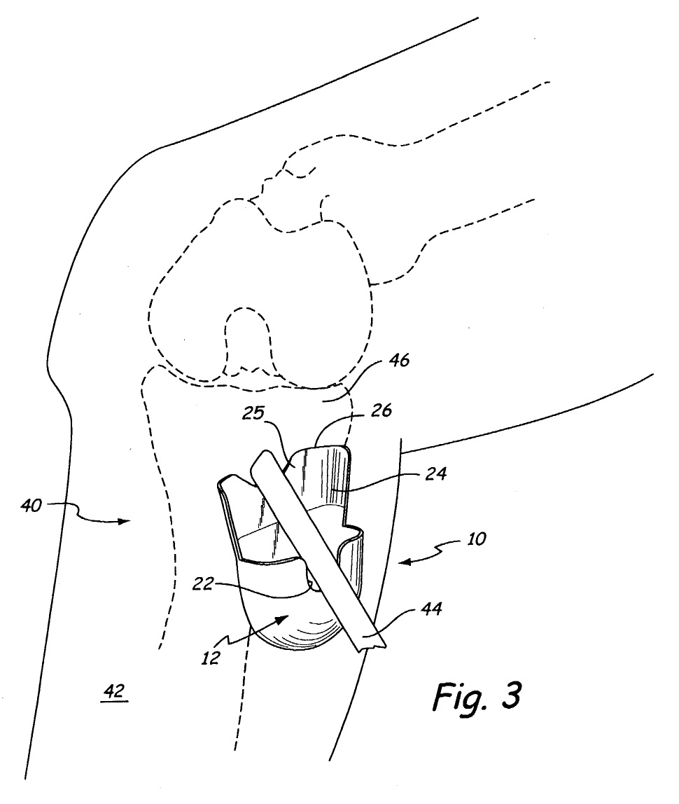

As illustrated in FIG. 3, the bone collection device 10 is manually held by the surgeon (not shown) and/or an operating room assistant (not shown) against the leg 40 on the external surface 42 so that the collecting surface, scoop wall 24, is held firmly just below the reamer 44. The tibia 46 is illustrated in broken lines. The scoop wall 24 is flexible such that it conforms to the surface 42 of the leg 40. However, alternatively, if the device of the present invention did not include the scoop wall 24, but instead just the chamber, the chamber wall could also be sufficiently flexible to conform to the surface of the leg. As the surgeon (not shown) creates the tibial tunnel with a standard rigid reamer, bone reamings are collected and pulled toward the external surface and into the device. Once the reamer enters the “joint”, the saline or other fluid earlier pressurized by an inflow pump to create extension within the knee joint releases suddenly through the tibial tunnel. Saline and reamings flush rapidly from the tunnel, down the scoop wall 24 and into the collection chamber 12. As the saline immediately begins to drain out of the collection chamber 12 including via the openings 32 along the bottom 20 of the collection chamber 12, the majority of the reamings are retained. Since they are denser than the fluids, the reamings settle to the bottom of the collection chamber but do not flow through the openings 32.

The majority of the fluid in the collection chamber 12 is then slowly poured over either side 18A or 18B with care taken not to pour out the bone graft reamings. The collection device 10 is set down with back face 16 facing downwardly and placed on or pressed against a sponge to permit remaining fluids to continue draining and wicking through the openings 32. As illustrated in FIG. 5, the reamings are maintained in the collection chamber 12, because of the obtuse angle between the scoop wall and the back face 16 of the chamber 12. This angled feature also helps the collection device 10 to continue to drain fluids when set down. Once the fluids have drained, the reamings can be used as graft material.

Fourth, the tibial guidepin is then placed through the tibial tunnel, up through the intraarticular area, through the femoral notch, and into the burred hole on the posterior aspect of the intercondylar notch at the femoral attachment of the ACL. The tibial guidepin is then pounded in a couple of millimeters. An acorn reamer is then brought in over the guidepin and the femoral tunnel is reamed up to the inner side of cortical bone.

As the saline inflow is pumping and a tunnel plug placed in the tibial tunnel to maintain fluid within the knee joint, an arthroscopic debrider blade is used to suction out and débride out any excess bone fragments from the femoral tunnel and knee joint. Other soft tissue debris is also débrided at this tine.

There are many known options for the femoral fixation and it is intended that all are included within the present invention. The method described herein is the endo button fixation. For this method, a guidepin is brought through the tibial tunnel into the femoral tunnel. The guidepin is then drilled through the outer cortex of the femur. An endo button reamer is then brought over the guidepin and a hole is reamed in the femoral cortex. A depth gauge is then utilized to measure the femoral tunnel length. This length is utilized to secure the endo button to the ACL graft.

Fifth, a passing pin is manually brought from the tibial side through the tibial tunnel, the intraarticular area and the femoral tunnel and carried through the skin over the femur. The B-T-B ACL autograft, harvested earlier, is then brought through the tibial and then into the femoral tunnels by placing sutures, which are in the endobutton, into the passing pin. The passing pin with sutures and graft is then pulled through both tunnels, the endo button hole, and quadriceps muscle and skin. Using the sutures the graft is pulled up into the joint, into the femoral tunnel with the endo button. It is advanced until the endobutton is pulled through the femoral cortex, thus the graft is in the anatomic position.

The endo button is flipped so it locks on the outer cortex of the femur. The graft is then pulled distally giving femoral fixation. When this is completed approximately 28 mm of patellar tendon from the graft are centered within the articular surface and the bone portions of the graft are within the bone tunnels of the femur and tibia respectively. The endo button is locked on the femoral side and distal traction can be placed on the graft with the sutures in the tibial side of the graft. The knee is brought into extension to ensure that there is no impingement on the graft within the femoral notch. At this point, a hole is drilled in the tibia just distal to the tibial tunnel. A screw with a washer is placed in the drilled hole. This is used as a post and the sutures which are on the tibial side within the bone plug of the ACL graft are then wrapped around the post while pulling traction and keeping the knee flexed approximately 30 degrees and putting posterior pressure on the tibia as these sutures are secured. The screw is tightened against the tibia to secure the graft. The incision area is then thoroughly irrigated with saline.

Sixth, skin and subcutaneous tissue retraction is then accomplished about the patella, thereby exposing the trough or pie-shaped area where the top portion of the autogenous bone-tendon-bone graft had been harvested in the early parts of the procedure. The present invention is then retrieved and the bone reamings earlier collected are used. In the typical case there are approximately 2-3 ml of bone reamings. These reamings are then used as grafting material for the previously created patellar defect. The interior surface of the chamber has a smooth and concave surface to facilitate effective retrieval and scooping out of the reamings. The reamings are scooped out of the collection device 12 by the surgeon using an operating room instrument such as the back end of an Adson's forceps and transferring the graft to the patellar defect. The surgeon compresses the reamings to conform to the defect in the patella. Usually all of the reamings are packed into the defect.

The peritenon which had been previously opened is then brought back over the defect. The peritenon is closed over the defect and the patella, which is now filled with graft material. Closure is continued distally over the patellar tendon. This ensures that the graft material will stay within the defect. The wounds are again irrigated. Two drains are then placed in two separate areas, one in the intraarticular area through the superolateral portal and one in the subcutaneous area where the ACL graft had been harvested. All cannulas are removed and the subcutaneous tissue is closed. The skin is closed. The wounds are sterilely dressed and a knee ranger brace is placed in full extension on the knee. Drains are utilized for 12-24 hours to drain fluid and blood that accumulate in the subcutaneous area and the knee joint. The tourniquet is deflated and the patient is then returned to recovery room.

This technique of tibial tunnel reaming, collection and retention, and use of the collected reamings as grafting material to repair the patellar defect is superior to other methods of either not treating the patellar defect or other methods of treating the patellar defect in bone-tendon-bone ACL reconstruction. If the patellar defect is not treated, the patient can feel the defect on their “kneecap” which can have a negative psychological effect. Most patients do not like the feel of that “hole” in their kneecap. The generally accepted practice among orthopedic surgeons is to treat the patellar defect because it is believed that the patella is weaker if not treated.

Using bone reamings is also superior to using a bone core because a bone core is rigid, is more difficult to work with, takes more time for a surgeon, operating room, and staff, for the OR procedure because it has to be sized, cut and formed to fit, is not pliable, and does not fit in the defect as well. Curetting tibial bone from the bottom of the tibia B-T-B graft harvest site also has the same time, difficulty, and resulting cost disadvantages as using a bone core; curetting bone also creates further morbidity to the tibia. It also is illogical to waste the reamings already available with the use of this device and create additional bone “chips” to use in the patella. Other methods using non-autogenous graft material are also more expensive.

The bone reamings provide greater surface area of bone which promotes healing, is more readily accepted by the patient's body, is more pliable for surgeons to use, saves time for the surgeon and OR staff, saves OR costs and time, and is more flexible and molds to the shape of the patellar defect. Prior art methods do not provide these advantages. The method of the present invention also provides use of autogenous grafting material, which optimizes healing and effective recovery for the patient. Use of autogenous material also reduces the risk of infection and disease transmission. This invention makes this technique efficient and effective for the surgeon and operating room staff.

Although the present invention has been described with reference to preferred embodiments, workers skilled in the art will recognize that changes may be made in form and detail without departing from the spirit and scope of the invention.

1. A collection device for collecting material during a surgical procedure, the device comprising:
a collection chamber defined by an encompassing wall and having an opening sufficient for permitting collection of the material during the surgical procedure; and
a scoop wall extending from the encompassing wall and having a distal end portion for positioning proximate a source of the material and for guiding the material to the collection chamber.

2. The device of claim 1 wherein the scoop wall includes a middle wall portion and sidewall portions extending from opposing sides of the middle wall portion in a generally curved manner to form a channel for guiding the material to the opening.

3. The device of claim 1 wherein the distal end portion of the scoop wall is sufficiently flexible to substantially conform to a surface proximate a source of the collecting material.

4. A collection device for collecting a fluid containing material, the fluid containing material being produced during a surgical procedure, the device comprising:
a collection chamber defined by an encompassing wall and an opening of a size and configuration for collection of the fluid containing material; and
at least one aperture within the collection chamber of a size to permit flow of the fluid from the chamber while retaining the fluid containing material within the chamber.

5. The collection device of claim 4 and further comprising a plurality of apertures disposed within the encompassing wall.

6. The device of claim 4 and further including a scoop wall extending from the encompassing wall of the collection chamber, the scoop wall having a distal end portion for positioning proximate a source of the fluid containing material.

7. The device of claim 6 wherein the scoop wall includes a middle wall portion and sidewall portions extending from opposing sides of the middle wall portion in a generally curved manner to form a channel for guiding the fluid containing material to the opening.

8. The device of claim 6 wherein the distal end portion of the scoop wall has a back surface of a configuration for positioning against a source of the fluid containing material.

9. The device of claim 6 wherein the distal end portion of the scoop wall is sufficiently flexible to substantially conform to a surface proximate a source of the fluid containing material.

10. The device of claim 4 wherein the encompassing wall of the collection chamber is sufficiently flexible to conform against a surface from which the fluid containing material is collected.

11. The device of claim 6 wherein the scoop wall is disposed at an obtuse angle with respect to the encompassing wall such that the device when placed on a surface, fluid drains through the at least one aperture.

12. The device of claim 6 wherein the scoop wall is disposed at an obtuse angle with respect to the encompassing wall such that the device when placed on a surface prevents the material within the fluid from flowing out of the opening.

13. A collection device for collecting surgical material during a surgical procedure, the device comprising:
a collection chamber defined by a wall having a distal portion sufficiently pliant to generally conform to the body proximate to the surgical site from which the surgical material is being collected and positionable proximate a source of the surgical material such that the surgical material is collected within the chamber.

14. The device of claim 13 wherein the surgical material includes bone chips collected in a solution during the surgical procedure, and the collection device further comprising at least one aperture disposed within the wall, the aperture being of a size that permits fluid to flow from the collection chamber while retaining bone chips therein.

15. The device of claim 14 and further including a plurality of apertures disposed within the wall, each aperture being of a size that permits flow of fluid from the chamber while retaining bone chips therein.

16. A device for use in a surgical procedure during which a reamer is used for tunneling within a bone, the device comprising:
a cup having a wall structure with a front wall and a back wall and an opening therebetween and a collection chamber positioned below the opening, and a cutout positioned proximate the top portion of the front wall of a size sufficient for positioning a reamer therein such that bone chips produced by the reamer fall within the collection chamber.

17. The device of claim 16 wherein the back wall is longer than the front wall.

18. The device of claim 17 wherein the back wall includes a middle wall portion and sidewall portions extending from opposing sides of the middle wall portion in a generally arcuate manner to form a channel for guiding the bone chips to the opening.

19. The device of claim 17 wherein the distal end portion of the back wall is sufficiently flexible to substantially conform to a surface proximate a source of the bone chips.

20. A method of collecting solid surgical materials in a fluid during surgery from a surgical location, the method comprising:
depositing the surgical materials in the fluid into a containment device having a cavity with at least one aperture and permitting the fluid to drain the cavity via the at least one aperture.

21. The method of claim 20 wherein the containment device includes a wall and where the method further includes positioning the wall at a location below the surgical location such that the surgical materials are deposited on the wall and are fed gravitationally to the containment device.

22. The method of claim 20 wherein the containment device is defined by an encompassing wall with a scoop wall extending therefrom and disposed at an obtuse angle with respect to the encompassing wall and the method further including positioning the containment device on a horizontal surface to permit further draining of the fluid from the cavity.

23. The method of claim 20 wherein the containment device is defined by an encompassing wall with a scoop wall extending therefrom and disposed at an obtuse angle with respect to the encompassing wall and the method further including positioning the containment device on a horizontal surface to retain the surgical material until the fluid substantially drains from the cavity.

24. The method of claim 21 wherein the surgical location is a hole being reamed in the bone of a patient and the solid surgical materials are bone chips, and wherein the method further includes utilizing the bone chips after drainage of the fluid as graft material in a surgical reconstruction of an anterior crucial ligament.

25. A collection device for collecting material during a surgical procedural, the device comprising:
a collection chamber defined by an encompassing wall and having an opening sufficient for permitting collection of the material during the surgical procedure, the collection chamber having a smooth concave inner surface to facilitate the effective retrieval of the material collected during the surgical procedure.

26. A collection device for collecting surgical material from a surgical site, the collection device having sufficient width that when positioned below the surgical site, a major portion of the surgical material generated at the surgical site is deposited within the collection device.
